(19) United States
(12) Patent Application Publication
(10) Pub. No.: US 2006/0235368 A1
(43) Pub. Date: Oct. 19, 2006

(54) Piston-actuated endoscopic tool

(75) Inventor: Dan Oz, Yehuda (IL)

Correspondence Address: BRUCE E. LILLING;LILLING & LILLING P.C., P.O. BOX 560, GOLDEN BRIDGE, NY, 10526, US

(73) Assignee: Sightline Technologies Ltd., Haifa (IL)

(21) Appl. No.: 10/527,249

(22) PCT Filed: Sep. 18, 2003
(86) PCT No.: PCT/IL03/00751

§ 371 Date: Mar. 9, 2005

Related U.S. Application Data
(60) Provisional application No. 60/414,741, filed on Sep. 30, 2002.

(51) Int. Cl. A61B 17/00; A61B 17/26; A61B 17/24

(52) U.S. Cl. 606/1; 606/113; 606/205

(57) ABSTRACT

Endoscopic apparatus is provided, having a distal end (102) for insertion into a body of a patient and a proximal end that is held outside the body of the patient. The apparatus includes a proximal cylinder (404), disposed in a vicinity of the proximal end of the endoscopic apparatus. A proximal piston (406) is slidably contained within the proximal cylinder. A distal cylinder (328) is disposed in a vicinity of the distal end of the endoscopic apparatus, and a distal piston (310) is slidably contained within the distal cylinder. A tube (402) for containing a liquid is coupled between the proximal and distal cylinders. A tool (e.g., biopsy tool 115) is coupled to be actuated by displacement of the distal piston, so as to perform a mechanical action on tissue of the body or contents of the body, responsive to displacement of the distal piston.

### FIELD OF INVENTION

The present invention relates generally to actuation of tools for flexible medical devices, and specifically to methods and devices for actuating endoscopic tools during medical procedures.

### BACKGROUND OF INVENTION

The use of an endoscope for examining a body cavity is well known in the art. The diagnostic and therapeutic advantages conferred by direct examination of the gastrointestinal tract with a flexible endoscope have made this method a standard procedure of modem medicine. One of the most common endoscopic procedures is colonoscopy, which is performed for a wide variety of purposes, including diagnosis of cancer, determination of the source of gastrointestinal bleeding, viewing a site affected by inflammatory bowel disease, removing polyps, and reducing volvulus and intussusception.

Flexible endoscopes typically include working channels, which run the length of the endoscope. One of the uses of these channels is to pass tools through the endoscope for performing diagnostic and therapeutic procedures within the body. Such tools include, for example, miniature biopsy forceps, which are passed through the channel and extend out through the distal end of the endoscope to take biopsy samples from the area under examination. Such tools are commonly controlled by means of cables or wires passing through a sheath, behind the tool itself, to the distal end of the endoscope. Tension is applied by a physician at the proximal end to the cables or wires, in order to induce a desired action of the tool at the distal end.

The extent to which the tool can be actuated by this technique is limited by friction between each wire and a sheath surrounding the wire. In particular, if a physician needs to overcome only a single turn in the gastrointestinal tract, then the force F1 that must be applied at the proxin al end in order to generate a force F2 at the distal end can be approximated as F1=F2*ₑμα, where μ is the coefficient of friction between the wire and the sheath, and a is the effective angle defmed by the turn in the gastrointestinal tract. If, as is conunon, the endoscope travels through a number i of turns αᵢ i in the gastrointestinal tract, then the total force can increase significantly (and often prohibitively) to F1=F2*ₑ^{μΣ|α}ᵢ^{|.}

To overcome the effects of friction incurred using wire-in-sheath based systems, attempts have been made to introduce hydraulics to endoscopes, but none of these have been commercially viable. All such hydraulic systems known to the inventor are complicated, expensive, bulky and/or require external power or pressure sources, as well as the equipment to manage these sources. Because of these drawbacks, only wire-based techniques are currently used for endoscopic steering and tool-control applications.

U.S. Pat. No. 5,569,299 to Dill et al., which is incorporated herein by reference, describes an endoscopic urological biopsy forceps with one stationary jaw and one moveable jaw, wherein the moveable jaw is actuated by a wire that runs internal to a hollow tube supporting the two jaws. The forceps is operated via actuation of the wire at the proximal end by a healthcare professional.

U.S. Pat. No. 5,431,645 to Smith et al., which is incorporated herein by reference, describes techniques for remote activation of endoscopic tools by various types of power sources including electric, mechanical, hydraulic and pneumatic sources located near the proximal end of the endoscope.

U.S. Pat. No. 5,779,646 to Koblish et al., which is incorporated herein by reference, describes a deflectable biopsy catheter wherein control wires running from the proximal to the distal end of the catheter are used to deflect the distal tip and/or activate the biopsy jaws. The control wires are attached to a piston, which is seated in a cylinder contained in a handle at the proximal end of the catheter, such that movement of the piston allows the operator to control the deflection of the distal tip and/or to activate the biopsy jaws.

U.S. Pat. No. 5,674,205 to Pasricha et al., which is incorporated herein by reference, describes a device for delivering a drug to a site within a lumen oof the body. The device resembles an elongated syringe with a distal piston/needle device containing a dose of drug, wherein a physician-operated end of the syringe is used to actuate the distal device via a fluid-filled tube connecting the distal and proximal ends.

U.S. Pat. No. 6,059,719 to Yamamoto et al., which is incorporated herein by reference, describes an endoscope system which contains a plurality of endoscope modules having different treatment instruments mounted therein, wherein the various treatment modules are freely exchangeable. In some embodiments, requisite forces to actuate the treatment modules are supplied via a transmission wire that traverses the length of the endoscope. Other embodiments, contain a liquid filled channel connecting a distal piston/cylinder arrangement to a proximal means of delivering fluid pressure, so as to move the distal piston. A transmission wire connects the distal piston to a treatment module such that movement of the distal piston actuates the treatment module.

A paper by Peirs et al., entitled, “A Micro Robotic Arm For A Self Propelling Colonoscope,” published in Proc. Actuator 98, 6th Int. Conf. on New Actuators, pp. 576-579, June 1998, which is incorporated herein by reference, describes a self-propelling endoscopic system for colonoscopy that comprises a flexible arm, which is controlled by shape memory alloy materials, to which are attached endoscopic tools. The endoscopic tools are controlled by either heating/cooling of shape memory alloy mechanisms, or by hydraulic means via a distal piston/cylinder apparatus. A simple piston/cylinder apparatus is used with a single pressure port on the cylinder, such that both positive and negative pressures must be used to operate an attached tool.

### SUMMARY OF THE INVETION

It is an object of some aspects of the present invention to provide an improved system and method for actuating a tool within a lumen.

It is a further object of some aspects of the present invention to provide an improved mechanism for actuating a tool within a body cavity of a patient for purposes of examination, diagnosis, or treatment.

It is still a further object of some aspects of the present invention to provide an improved mechanism for actuating a tool within a body cavity of a patient for purposes of obtaining a tissue biopsy or performing another procedure.

In preferred embodiments of the present invention, an endoscopic tool for performing a mechanical action on tissue or contents of the gastrointestinal tract of a patient or within another body cavity, is advanced through a channel in a flexible endoscope placed in the cavity. The endoscopic tool is brought into proximity with a target (e.g., tissue, an intestinal calculus, or a stone), and is actuated with the aid of an actuation mechanism. coupled to the tool, near the distal end of the channel, to perform a mechanical action on the target. The actuation mechanism comprises one or more cylinders, each containing a piston, whereby movement of the pistons actuates a linkage, coupled to the tool, causing the tool to function. Movement of the pistons is achieved by introducing liquid into or removing liquid from the corresponding cylinders. The liquid is delivered from the proximal end of the endoscope to the cylinders of the actuation mechanism near the distal end of the endoscope via a closed system of one or more flexible tubes, passing through the working channel. These embodiments of the present invention obviate the need for tool-actuating wires running the length of the endoscope, thus minimizing difficulties, such as friction, which are commonly associated with wire-based actuation.

Preferably, the tool is actuated by pressurizing the pistons in the actuation mechanism, by driving fluid under pressure into the pistons, rather than by withdrawing fluid from the pistons as in hydraulically-actuated tools known in the art. In the context of the present patent application and in the claims, “actuating” the tool refers to perfomiring an operation requiring force to be exerted by the tool, such as closing a biopsy forceps. In actuation mechanisms based on withdrawing fluid from a hydraulic mechanism, only one atmosphere of negative pressure can be applied, so that forces applied by the tool are limited. When liquid is driven into the pistons under positive pressure, much greater forces can be applied.

Means for providing liquid to the cylinders in the actuation mechanism via the flexible tubes are preferably located near the proximal end of the endoscope, external to the patient. In a preferred embodiment of the present invention, a drive-piston/cylinder system is used to provide pressure to the liquid in the flexible tubes, so as to drive the actuation mechanism. Preferably, the operator uses hand and/or foot movements to displace one or more drive pistons in their respective cylinders, resulting in movement of liquid into or out of the actuation mechanism cylinders, and thus movement of the corresponding pistons and the desired actuation of the tool near the distal end of the endoscope. Thus, physical forces applied by the operator are directly or proportionately applied to actuate the endoscopic tool, providing the operator with a sense of feedback. After a relatively short training and practice period, the operator typically learns the amount of force necessary to apply to a mechanical user-interface device such as a joystick, in order to operate the tool during a particular procedure. Leveraging, or other aspects of the mechanical and/or hydraulic design of the actuation mechanism, control the physical force required to actuate the tool.

In a preferred embodiment of the present invention, each actuation mechanism cylinder comprises one port for introduction or withdrawal of liquid so as to move the corresponding piston. The pistons divide each actuation mechanism cylinder into two regions: (a) a liquid-transfer region, comprising a port through which liquid is actively added or withdrawn, and (b) a passive region, which may be open at one end, or which may comprise a spring or a fixed amount of a compressible fluid such as air. Preferably, the actuation mechanism cylinder is aligned with the longitudinal axis of the endoscope, and the liquid-transfer region is closer than the passive region to the distal end of the endoscope. This arrangement is preferred for some applications, because when liquid is added to the distal end of one of the actuation mechanism cylinders, a tensile force develops in members of the actuation mechanism that connect the piston to the tool, reducing the possibility of buckling of the thin members due to compressive loads. Mechanical linkages between two or more of the actuation mechanism cylinders are preferably designed so as to maintain tensile loads in these actuation mechanism members when liquid is added to the liquid-transfer regions of one or more of the cylinders. Alternatively or additionally, one or more suitably-configured rods are coupled to the actuation mechanism cylinders so as to be placed in compression during application or removal of liquid in the liquid-transfer region of the cylinder(s), and to thereby facilitate actuation of the tool.

For applications in which the passive region of each actuation mechanism cylinder contains a compressible fluid (e.g., air), the fluid typically functions essentially as a spring, and acts to return the piston to its equilibrium position. Alternatively or additionally, this region comprises a solid spring to assist in returning the piston to its equilibrium position once no external pressure is applied to tle cylinder.

In another preferred embodiment of the present invention, each actuation mechanism cylinder comprises two ports, one on each side of the piston, which are coupled respectively to two liquid-transfer regions of the cylinder, into or out of which liquid is actively added or removed. Flexible tubes convey hydraulic pressure from the proximal end of the endoscope to each port Movement of a given piston in the actuation mechanism is initiated responsive to the difference in the pressure on opposing sides of the piston. By regulating the pressure on each side of the piston, accurate control of the force delivered by the piston to the actuation mechanism linkage is achieved. Preferably, the pressure is positive on both sides of the piston, during respective periods of actuation of the tool.

There is therefore provided, in accordance with an embodiment of the present invention, endoscopic apparatus having a distal end for insertion into a body of a patient and a proximal end that is held outside the body of the patient, the apparatus including:

a proximal cylinder, disposed in a vicinity of the proximal end of the endoscopic appa-atus;

a proximal piston, slidably contained within the proximal cylinder;

a distal cylinder, disposed in a vicinity of the distal end of the endoscopic apparatus;

a distal piston, slidably contained within the distal cylinder;

a tube for containing a liquid, coupled between the proximal and distal cylinders; and

a tool coupled to be actuated by displacement of the distal piston, so as to perform a mechanical action on tissue of the body or contents of the body, responsive to displacement of the distal pistonl

In an embodiment, the tool, the distal cylinder, the distal piston and the tube are adapted to be passed through a working channel of an endoscope so as to access a region within the body using the endoscope.

In an embodiment the tool is adapted to access a portion of a gastrointestinal tract of the patient.

For some applications, the tool includes a biopsy tool. Alternatively or additionally, the tool includes a therapeutic tool.

In an embodiment:

(a) the distal cylinder has two regions, on respective sides of the distal cylinder,

(b) the tube is adapted to be in communication with a first one of the regions,

(c) a second one of the regions is configured such that motion of the distal piston in a first direction changes a fluid pressure in the second region, and

(d) the distal piston is coupled to the distal cylinder so as to experience a force in a second direction, opposite to the first direction, responsive to the change in fluid pressure.

For some applications, the proximal piston is adapted to be hand operated. In an embodiment, the apparatus includes a linkage, coupled to the proximal piston, which is adapted to facilitate hand operation of the proximal piston.

For some applications, the tool is coupled to the distal piston so as to be actuated by pressurization of the tube by the liquid due to operation of the proximal piston. For example, the tool may include a forcers, anld actt]ating the tool by pressurization of the tube causes the forceps to close. Alternatively, the tool includes a snare, and actuating the tool by pressurization of the tube causes the snare to close.

There is further provided, in accordance with an embodiment of the present invention, endoscopic apparatus having a distal end for insertion into a body of a patient and a proximal end that is held outside the body of the patient, the apparatus including:

a distal piston;

a distal cylinder within which the distal piston is slidably contained, and which is in a vicinity of the distal end of the endoscopic apparatus, the distal cylinder having a first distal port proximal to the distal piston and a second distal port distal to the distal piston;

a tool coupled to be actuated by displacement of the distal piston;

a proximal piston;

a proximal cylinder within which the proximal piston is slidably contained, and which is in a vicinity of the proximal end of the endoscopic apparatus, the proximal cylinder having a first proximal port proximal to the proximal piston and a second proximal port distal to the proximal piston; and

first and second tubes, the first tube coupling one of the proximal ports to one of the distal ports, and the second tube coupling the other one of the proximal ports to the other one of the distal ports, such that:

(a) proximal motion of the proximal piston drives liquid through one of the tubes to apply a positive pressure to a first side of the distal piston to displace the distal piston in a first direction and actuate the tool to be in a first state, and

(b) distal motion of the proximal piston drives liquid through the other one of the tubes to apply a positive pressure to a second side of the distal piston to displace the distal piston in a second direction and actuate the tool to be in a second state.

In an embodiment, the tool, the distal cylinder, the distal piston and the tube are adapted to be passed through a working channel of an endoscope so as to access a region within the body using the endoscope.

In an embodiment, the tool is adapted to access a portion of a gastrointestinal tract of the patient

For some applications, the tool includes a biopsy tool and/or a therapeutic tool.

In an embodiment, the proximal piston is adapted to be hand operated. For example, the apparatus may include a linkage, coupled to the proximal piston, which is adapted to facilitate hand operation of the proximal piston.

There is still further provided, in accordance with an embodiment of the present invention, endoscopic apparatus having a distal end for insertion into a body of a patient and a proximal end that is held outside the body of the patient, the apparatus including:

first and second proximal cylinders, disposed in a vicinity of the proximal end of the endoscopic apparatus;

first and second proximal pistons, slidably contained within the respective proximal cylinders;

at least one distal cylinder, disposed in a vicinity of the distal end of the endoscopic apparatus;

at least one distal piston, slidably contained within the at least one distal cylinder;

a first tube for containing a liquid, coupled between the first proximal cylinder and the at least one distal cylinder;

a second tube for containing a liquid, coupled between the second proximal cylinder and the at least one distal cylinder;

a mechanical linkage, coupled to the first and second proximal pistons so as to:(a) move the first proximal piston and cause positive pressure in the first tube when the mechanical linkage is displaced in a first direction, and (b) move the second proximal piston and cause positive pressure in the second tube when the mechanical linkage is displaced in a second direction; and

a tool coupled to be actuated by displacement of the at least one distal piston, so as to perform a mechanical action on tissue of the body or contents of the body, responsive to displacement of the distal piston.

The present invention will be more fully understood from the following detailed description of the preferred embodiments thereof, taken together with the drawings, in which:

### BRIEF DESCRIPTION OF TIE DRAWINGS

FIG. 1 is a schematic, sectional drawing of an endoscopic tool comprising a hydraulic actuation mechanism, according to a preferred embodiment of the present invention;

FIG. 2 is a schematic, sectional drawing of an endoscopic tool comprising a hydraulic actuation mechanism, according to another preferred embodiment of the present invention;

FIG. 3 is a schematic, sectional drawing of an endoscopic tool comprising a hydraulic actuation mechanism, according to yet another preferred embodiment of the present invention;

FIG. 4 is a schematic, sectional drawing of an endoscopic tool comprising a hydraulic actuation mechanism, according to still another preferred embodiment of the present invention;

FIG. 5 is a schematic, sectional drawing of an endoscopic tool comprising a hydraulic actuation mechanism, according to a further preferred embodiment of the present invention; and

FIG. 6 is a schematic, sectional drawing of an endoscopic tool comprising a hydraulic actuation mechanism, according to yet a further preferred embodiment of the present invention.

### DETAILED DESCRIPTION OF PREFERRED EMBODIMENTS

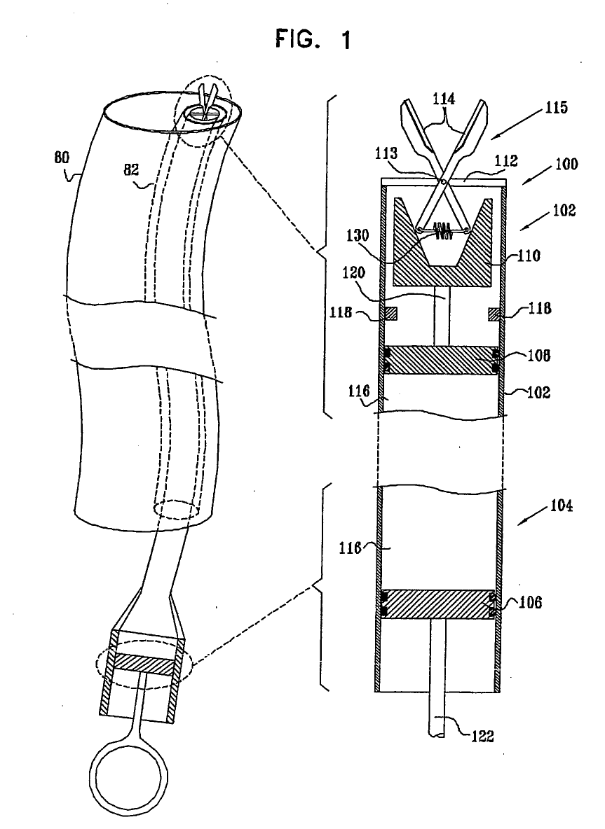

Reference is now made to FIG. 1, which is a schematic sectional drawing of a flexible endoscopic device 100 comprising a hydraulically-actuated tool, in accordance with a preferred embodiment of the present invention. Endoscopic device 100 comprises a distal portion 102, which is advanced through a working channel 82 of an endoscope 80 placed in the gastrointestinal tract of a patient. Device 100 additionally comprises a proximal portion 104, part of which remains external to the patient and is accessible to the operator of the tool. Typically, the tool comprises a biopsy tool 115, comprising two opposable biopsy jaws 114. Tool 115 is disposed near the distal tip of device 100, for excising or sampling tissue inside the gastrointestinal tract.

Means for actuating tool 1 15 are located near the distal tip of portion 102. In a preferred embodiment, biopsy jaws 114 each comprise a spoon-shaped lever and rotate about a common pivot point 113, such that the spoon portion of each lever is able to grab and dissect tissue. Pivot point 113 is coupled to an end cap 112, which is coupled to the distal tip of device 100. Movement of a wedge-shaped member 110 actuates jaws 114, such that distal motion of member 110, i.e., motion in a distal direction (upward in the view shown in the figure), causes closing of the biopsy jaws, while proximal motion of member 110, i.e., motion in the proximal (downward) direction, allows the biopsy jaws to open. In a preferred embodiment, tool 115 comprises a spring 130, which acts to open biopsy jaws 114 when member 110 moves proximally.

Mechanical stops 118 are preferably coupled to the inside of portion 102, distal to piston 108, to limit the motion of the piston when positive pressure is applied. Typically, when device 100 is advanced through working channel 82, pressure is applied to piston 108 so as to press piston 108 against stops 118 and maintain jaws 114 in the closed position.

Member 110 is coupled to a distal piston 108 by a rod 120, such that movement of piston 108 causes an equal movement of member 110. Preferably, rod 120 has a length to diameter ratio that is relatively small (for example less that 10), such that rod 120 can transmit compressive loads without buckling or appreciable bending. Alternatively, rod 120 may be absent, such that piston 108 is directly fixed to member 110.

Actuation of tool 115 is achieved by movement of a proximal piston 106, which varies the pressure of a liquid-filled duct 116, intermediate to piston 106 and piston 108, so as to control the force acting on piston 108. Preferably, duct 116 is filled with a substantially-incompressible biocompatible liquid (for example water or saline solution). Pressurizing duct 116 using piston 106 drives piston 108 in the distal direction, thereby closing jaws 114. The operator initiates movement of piston 106 via a mechanical linkage 122, which is coupled to piston 106 and accessible near the distal end of portion 104. In a preferred embodiment, linkage 122 is a simple rod, whose motion is directly imparted to piston 106. Alternatively, linkage 122 comprises a joystick, wheel, or other mechanism to improve the ease of use of the tool, for example by reducing the force required of the operator. It is noted that use of proximal piston 106 eliminates the more complex proximal pressure apparatus required by hydraulic endoscopic biopsy tools known in the art.

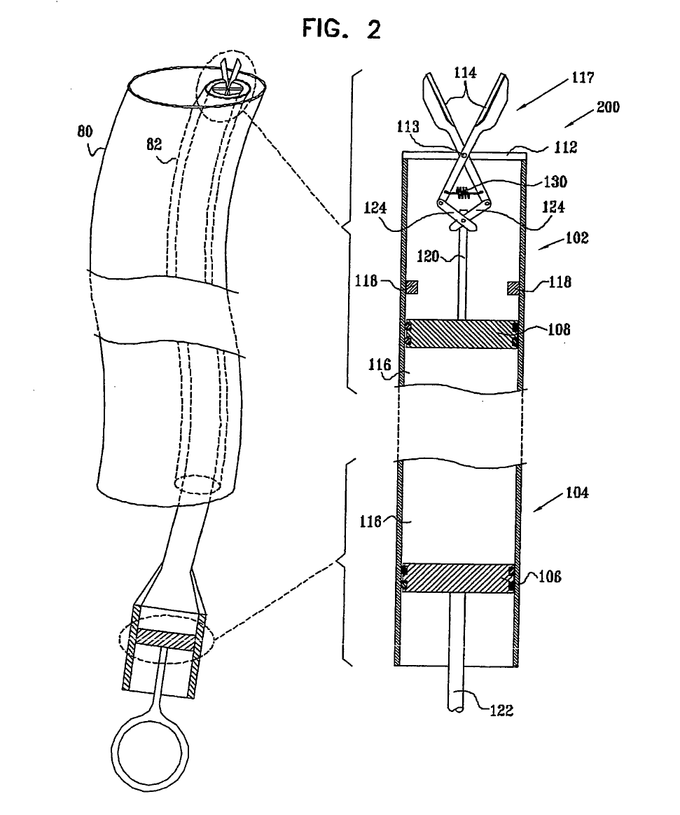

FIG. 2 is a schematic sectional drawing of a flexible endoscopic device 200 comprising a hydraulically-actuated tool, in accordance with a preferred embodiment of the present invention. Device 200 generally functions in a manner similar to that of device 100 described hereinabove with reference to FIG. 1, but comprises different mechanism for transferring motion of piston 108 into actuation of a biopsy tool 117.

Movement of piston 108, due to pressure in duct 116, is transferred to rod 120, to linkage members 124, and to the proximal end of biopsy jaws 114. In this manner, distal motion of piston 108 tends to open jaws 114 and to stretch spring 130, while proximal motion of piston 108 tends to close jaws 114 with the assistance of spring 130. Closingjaws 114 of biopsy tool 117 thus induces tension in rod 120 and linkage members 124, minimizing the possibility of buckling of these parts. Additionally, by modifying the size of the elements in the linkage of tool 117, the force applied by biopsy jaws 114 can be regulated to be a desL cd multiple of the force applied to piston 106.

It is noted that use of proximal piston 106 eliminates the more complex proximal pressure apparatus required by hydraulic endoscopic biopsy tools known in the art.

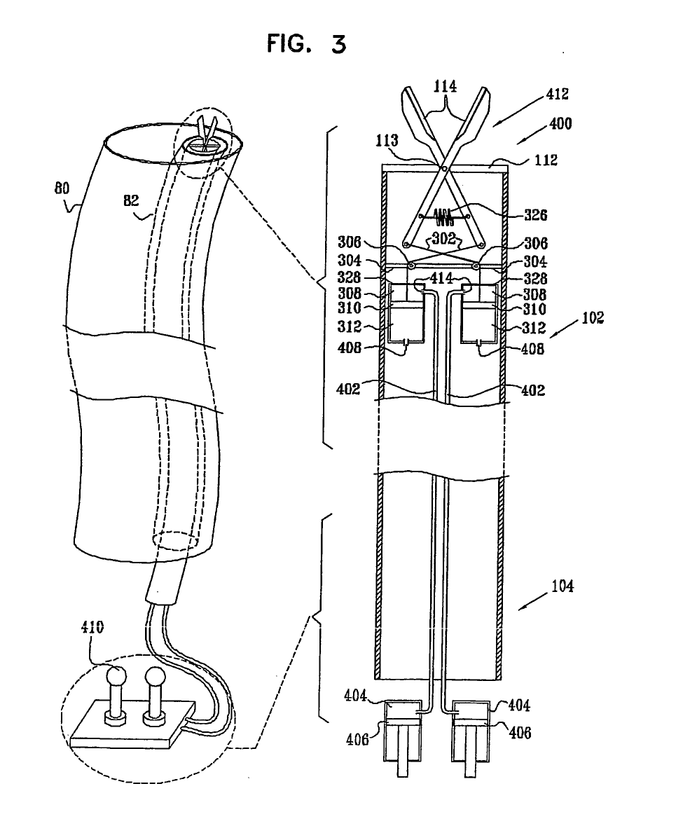

FIG. 3 is a schematic sectional drawing of a flexible endoscopic device 400 comprising a hydraulically-actuated tool 412, in accordance with a preferred embodiment of the present invention. Means for facilitating operation of the tool are located in portion 102, and preferably comprise a plurality of cylinders 328, each of which having disposed therein a piston 310. Advantageously, a plurality of cylinders 328 provides the physician with the ability to independently control either one of jaws 114, e.g., so as to be able to operate tool 412 off of the center line of endoscopic device 400. It will be appreciated that other endoscopic tools or sets of tools (not shown) used in other applications also benefit from the increased degrees of freedom provided by a plurality of cylinders 328.

Each piston 310 is preferably coupled to one end of respective wires 302. The opposite ends of wires 302 are coupled to respective portions of biopsy jaws 114. In a preferred embodiment, a crosspiece 304, which is coupled to portion 102, comprises a plurality of pulleys 306, so as to route wires 302 between pistons 310 and jaws 114.

Movement of pistons 310 is driven by liquid delivered to or withdrawn from cylinders 328 via flexible tubes 402. Preferably, each cylinder 328 is aligned parallel to the longitudinal axis of the endoscope, and liquid is delivered to or withdrawn from a port 414 near the distal end of the cylinder. Each cylinder is thus divided into two sections by piston 310: (a) a liquid transfer section 308, closer to the distal end of portion 102, where liquid is delivered or withdrawn, and (b) a passive section 312, closer to the proximal end of portion 102.

A spring 326 is preferably coupled to biopsy jaws 114, so as to tend to open the jaws. There is thus minimal or no use of suction applied to cylinders 328 to move pistons 310 distally and open jaws 114. This reduced use of suction decreases potential problems associated with collapse of flexible tubes 402. Also, suction as a means for generating useful motion of the endoscope is generally limited to one atmosphere, while positive pressure can exceed one atmosphere. Experiments performed using th prciples of he present nr h av enerated pressures of 50 atmospheres at the distal end, using only the force easily generated by hand, applied to the simple and inexpensive apparatus preferred in accordance with these embodiments of the present invention. It is emphasized that prior art systems for hydraulic endoscopic biopsy tools generally require complicated and expensive apparatus, which utilize pumps and pressure-regulation apparatus or other powered equipment to operate.

For some applications, passive section 312 of each cylinder 328 comprises an orifice 408, allowing a fluid (typically air) to enter or leave as piston 310 is displaced. In a preferred embodiment of the present invention, the passive section of cylinder 328 comprises an elastic element such as a spring, optionally replacing spring 326, which acts to maintain piston 310 in its equilibrium position. Alternatively, the passive section of cylinder 328 is sealed and encloses a compressible fluid such as air, which acts like a spring when piston 310 is displaced, returning the piston to its equilibrium position.

Liquid is delivered to or withdrawn from each cylinder 328 responsive to the operation of a corresponding drive-piston 406 in a drive-cylinder 404. Each drive-piston 406 is preferably coupled to the respective cylinder 328 by one of flexible tubes 402. Applying a distally-directed force to drive-piston 406 pressurizes the liquid in drive-cylinder 404. This pressure is transmitted through the liquid in tube 402 and in cylinder 328, and comes to act on piston 310, to cause actuation of tool 412 as described hereinabove. In particular, distal motion of pistons 406 causes closing of jaws 114, while expansion of spring 326 causes opening of jaws 114. The ratio of the driving force applied to drive-piston 406 to the pressure force received by piston 310 is generally proportional to the area ratio of the two piston faces. Thus, fine control of tool 412 can be achieved by decreasing the area of piston 406 relative to the area of piston 310. In this manner, operator-induced motions of piston 406 can be leveraged to yield fine motions of piston 310. The force required to actuate the tool can be selected by sizing drive-piston 406 and piston 310 appropriately.

In some preferred embodiments of the present invention, a mechanical linkage 410 such as a joystick mechanically coupled to pistons 406, is used to actuate drive-pistons 406 to make actuating the tool more ergonomic. For applications in which more cylinders are used at the distal and/or proximal ends of the en-udosc-ope, appropriate changes in the linkage are provided, so as to facilitate greater ease of use for the operator.

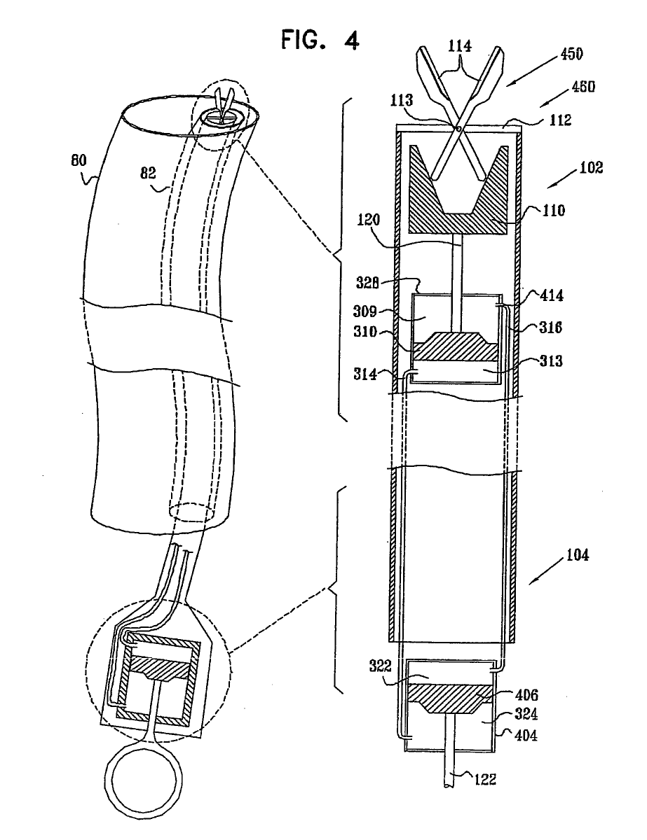

FIG. 4 is a schematic sectional drawing of a flexible endoscopic device 460 comprising a hydraulically-actuated tool 450, in accordance with a preferred embodiment of the present invention. As described hereinabove with reference to FIG. 1, movement of wedge-shaped member 110 actuates biopsy jaws 114, such that distal motion of member 110 causes closing of the biopsy jaws, while proximal motion allows the biopsy jaws to open. In contrast to some known hydraulic biopsy tool control apparatus, tool 450 preferably does not include a spring to open or close biopsyjaws 114.

Actuation of tool 450 is initiated by movement of drive-piston 406 controlled by the operator. Motion of drive-piston 406 varies the pressure in a distal flexible tube 316 and a proximal flexible tube 314; which, respectively, couple: (a) a distal drive-portion 322 of drive-cylinder 404 to a distal portion 309 of cylinder 328, and (b) a proximal drive-portion 324 of drive-cylinder 404 to a proximal portion 313 of cylinder 328. In this manner, fine control of the force acting on piston 310 is typically achieved. Preferably, tubes 314 and 316 are filled with a substantially-incompressible biocompatible liquid (for example water or saline solution).

The operator initiates movement of piston 406 via mechanical linkage 122, which is coupled to piston 406 and is accessible near the proximal end of portion 104. Advantageously, movement of piston 310 in both the proximal and the distal direction is achieved by application of positive pressure into tubes 316 and 314, respectively. In particular, proximal motion of drive-piston 406 closes jaws 114, and distal motion of drive-piston 406 opens jaws 114. Thus, the embodiment of the present invention shown in FIG. 4 performs active work in both directions in response to the application of positive pressure, typically without the use of a spring. Advantageously, high levels of positive hydraulic pressure are easily generated to both open and close biopsy jaws or to appropriately actuate other endoscopic tools.

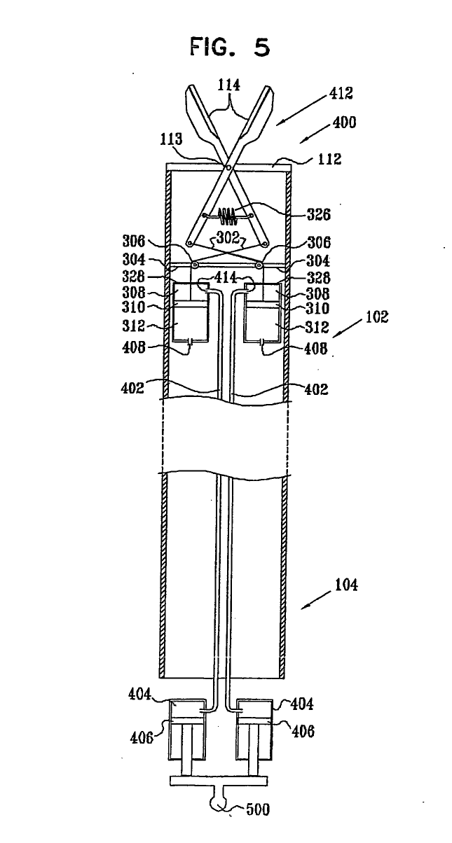

FIG. 5 is a schematic sectional drawing of flexible endoscopic device 400, comprising hydraulically-actuated tool 412, in accordance with a preferred embodiment of the present invention. The embodiment shown in FIG. 5 is generally similar to that shown in FIG. 3, except in that mechanical linkage 410 (which is shown in FIG. 3 as having two joysticks), is replaced in FIG. 5 by a single joystick 500. When joystick 500 is moved in one direction by a user, pressure in one of drive-cylinders 404 is increased, producing a corresponding increase in the pressure in the flexible tube 402 coupled thereto. When joystick 500 is moved in the other direction, pressure in the other one of drive-cylinders 404 is increased, producing a corresponding increase in the pressure in the flexible tube 402 coupled to that drive-cylinder.

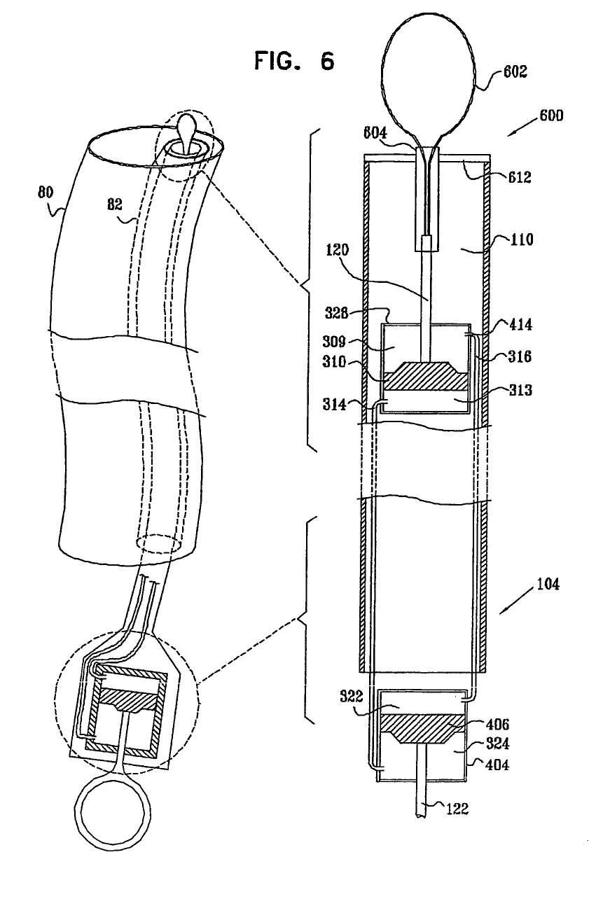

FIG. 6 is a schematic sectional drawing of a flexible endoscopic device 600 comprising a hydraulically-actuated snare 602, in accordance with a preferred embodiment of the present invention. Endoscopic device 600 is generally similar to endoscopic device 460, described hereinabove with reference to FIG. 4, except in that biopsy jaws 114 and related apparatus shown in FIG. 4 are replaced in the embodiment shown in FIG. 6 by snare 602. Snare 602 is typically used to surround a polyp or other portion of tissue of a patient. When the snare is gradually withdrawn into a casing 604 thereof, which is mounted to a distal end-piece 612 of endoscopic device 600, the tissue is thereby removed

Actuation of snare 602 is initiated by movement of drive-piston 406. Motion of drive-piston 406 varies the pressure in distal flexible tube 316 and proximal flexible tube 314, as described hereinabove. In this manner, fine control of the force acting on piston 310 is typically achieved. Motion of piston 310, in turn, is preferably directly converted to actuation (i.e., opening or closing) of snare 602. Opening and closing of snare 602 is thus typically achieved by application of positive pressure into tubes 314 and 316, respectively.

It will be appreciated that snare 602 could be replaced by a retractable forceps or other medical tools kmown in the art.

In a preferred embodiment of the present invention, techniques described herein are applied in conjunction with methods and apparatus described in a co-pending U.S. provisional patent application, tentative serial No. 60/395,694, entitled, “Piston-actuated endoscopic steering system,” filed Jul. 11, 2002, which is assigned to the assignee of the present patent application and is incorporated herein by reference. That patent application states:
“In preferred e-rbodiment of the present invention, a distal section of a flexible endoscope is advanced through the gastrointestinal tract with the aid of a steering mechanism near the distal end of the endoscope. The steering mechanism comprises one or more cylinders, each containing a piston, wherein movement of one or more of the pistons actuates rods, wires and/or cables in the steering mechanism to cause turning of the distal end of the endoscope. Movement of the one or more pistons is achieved by introducing or removing fluid into/from the corresponding cylinders, so as to cause a motion of the piston. The fluid is delivered from the proximal end of the endoscope to the cylinders of the steering mechanism near the distal end of the endoscope via a closed system of flexible tubes.”

Alternatively or additionally, techniques described herein are applied in conjunction with methods and apparatus described in PCT Patent Publication WO 00/44275, entitled, “Propulsion of a probe in the colon using a flexible sleeve,” and U.S. Pat. application Ser. No. 09/646,941 in the national phase thereof, which are assigned to the assignee of the present patent application and are incorporated herein by reference. The '275 publication states:
“In preferred embodiments of the present invention, a probe containing an endoscopic instrument is advanced through the lower gastrointestinal tract of a patient by inflation of a flexible sleeve coupled to the probe. One end of the sleeve is anchored, typically at or adjacent to the patient's anus. As the sleeve is inflated, preferably using a pressurized gas, the probe is propelled forward, and the sleeve is fed out gradually between the probe and the anus.

The portion of the sleeve that is inflated expands radially outward and remains substantially stationary relative to the intestinal wall as long as it is inflated.

Longitudinal motion of the sleeve relative to the wall generally occurs only at and adjacent to the probe itself. The probe is thus advanced easily, and trauma to the gastrointestinal tract is minirnized. To remove the probe, the sleeve is deflated and is used to pull the probe back out through the anus. . . “In other preferred embodiments of the present invention, the sleeve is stored in a compact state, typically folded or rolled up, inside or immediately adjacent to the probe. Most preferably, the folded or rolled-up probe is stored in a recess in a proximal portion of the probe. As tie probe advances, the sleeve feeds gradually out of its stored state and expands against the intestinal wall. . . “In preferred embodiments of the present invention, advancing the probe through the gastrointestinal tract by way of inflating the sleeve reduces or eliminates the necessity of applying mechanical force at a proximal end of the probe (outside the patient's body) to insert the probe, as is required using conventional endoscopes. The present invention thus reduces or eliminates the necessity of applying concentrated, local pressure to any part the patient's body, reduces or eliminates rubbing and friction between the unit or parts of it and the patient's body, and avoids ejecting fluids or other materials into the body's passageway.”
In accordance with a preferred embodiment of the present invention, by combining the techniques of the present patent application with the techniques described in the “Piston-actuated endoscopic steering system” application and the “Propulsion of a probe in the colon using a flexible sleeve” application, an endoscope is provided which performs substantially all motions (i.e., tool-operation, steering and propulsion) without the need for wires or other elements which are known to sometimes apply undesired forces to the gastrointestinal tract and/or to generate excess friction forces during operation.

It will be appreciated that the preferred embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art. For example, although preferred embodiments of the present invention have been described herein with respect to a hydraulic tool for operation in the gastrointestinal tract, it will be appreciated that these techniques may be adapted for use in other body cavities as well.

1. Endoscopic apparatus having a distal end for insertion into a body of a patient and a proximal end that is held outside the body of the patient, the apparatus comprising:
a proximal cylinder, disposed in a vicinity of the proximal end of the endoscopic apparatus;
a proximal piston, slidably contained within the proximal cylinder;a distal cylinder, disposed in a vicinity of the distal end of the endoscopic apparatus;
a distal piston, slidably contained within the distal cylinder;
a tube for containing a liquid, coupled between the proximal and distal cylinders;
and a tool coupled to be actuated by displacement of the distal piston, so as to perform a mechanical action on tissue of the body or contents of the body, responsive to displacement of the distal piston.

2. Apparatus according to claim 1, wherein the tool, the distal cylinder, the distal piston and the tube are adapted to be passed through a working channel of an endoscope so as to access a region within the body using the endoscope.

3. Apparatus according to claim 1, wherein the tool is adapted to access a portion of a gastrointestinal tract of the patient.

4. Apparatus according to claim 1, wherein the tool comprises a biopsy tool.

5. Apparatus according to claim 1, wherein the tool comprises a therapeutic tool.

6. Apparatus according to claiml, wherein the distal cylinder has two regions, on respective sides of the distal cylinder, wherein the tube is adapted to be in communication with a first one of the regions, wherein a second one of the regions is configured such that motion of the distal piston in a first direction changes a fluid pressure in the second region, and wherein the distal piston is coupled to the distal cylinder so as to experience a force in a second direction, opposite to the first direction, responsive to the change in fluid pressure.

7. Apparatus according to claim 1, wherein the proximal piston is adapted to be hand operated.

8. Apparatus according to claim 7, wherein the apparatus comprises a linkage, coupled to the proximal piston, which is adapted to facilitate hand operation of the proximal piston.

9. Apparatus according to claim 1, wherein the tool is coupled to the distal piston so as to be actuated by pressurization of the tube by the liquid due to operation of the proximal piston.

10. Apparatus according to claim 9, wherein the tool comprises a forceps, and wherein actuating the tool by pressurization of the tube causes the forceps to close.

11. Apparatus according to claim 9, wherein the tool comprises a snare, and wherein actuating the tool by pressurization of the tube causes the snare to close.

12. Endoscopic apparatus having a distal end for insertion into a body of a patient and a proximal end that is held outside the body of the patient, the apparatus comprising:
a distal piston;
a distal cylinder within which the distal piston is slidably contained, and which is in a vicinity of the distal end of the endoscopic apparatus, the distal cyiinder having a first distaI port proximal to the distal piston and a second distal port distal to the distal piston;
a tool coupled to be actuated by displacement of the distal piston;a proximal piston ;
a proximal cylinder within which the proximal piston is slidably contained, and which is in a vicinity of the proximal end of the endoscopic apparatus, the proximal cylinder having a first proximal port proximal to the proximal piston and a second proximal port distal to the proximal piston; and first and second tubes, the first tube coupling one of the proximal ports to one of the distal ports, and the second tube coupling the other one of the proximal ports to the other one of the distal ports, such that:
(a) proximal motion of the proximal piston drives liquid through one of the tubes to apply a positive pressure to a first side of the distal piston to displace the distal piston in a first direction and actuate the tool to be in a first state, and
(b) distal motion of the proximal piston drives liquid through the other one of the tubes to apply a positive pressure to a second side of the distal piston to displace the distal piston in a second direction and actuate the tool to be in a second state.

13. Apparatus according to claim 12, wherein the tool, the distal cylinder, the distal piston and the tube are adapted to be passed through a working channel of an endoscope so as to access a region within the body using the endoscope.

14. Apparatus according to claim 12, wherein the tool is adapted to access a portion of a gastrointestinal tract of the patient.

15. Apparatus according to claim 12, wherein the tool comprises a biopsy tool.

16. Apparatus according to claim 12, wherein the tool comprises a therapeutic tool.

17. Apparatus according to claim 12, wherein the proximal piston is adapted to be hand operated.

18. Apparatus according to claim 17, wherein the apparatus comprises a linkage, coupled to the proximal piston, which is adapted to facilitate hand operation of the proximal piston.

19. Endoscopic apparatus having a distal end for insertion into a body of a patient and a proximal end that is held outside the body of the patient, the apparatus comprising:
first and second proximal cylinders, disposed in a vicinity of the proximal end of the endoscopic apparatus;
first and second proximal pistons, slidably contained within the respective proximal cylinders;
at least one distal cylinder, disposed in a vicinity of the distal end of the endoscopic apparatus;
at least one distal piston, slidably contained within the at least one distal cylinder;
a first tube for containing a liquid, coupled between the first proximal cylinder and the at least one distal cylinder;
a second tube for containing a liquid, coupled between the second proximal cylinder and the at least one distal cylinder; a mechanical linkage, coupled to the first and second proximal pistons so as to:
(a) move the first proximal piston and cause positive pressure in the first tube when the mechanical linkage is displaced in a first direction, and
(b) move the second proximal piston and cause positive pressure in the second tube when the mechanical linkage is displaced in a second direction; and
a tool coupled to be actuated by displacement of the at least one distal piston, so as to perform a mechanical action on tissue of the body or contents of the body, responsive to displacement of the distal piston.
